(19) United States
(12) Patent Application Publication
(10) Pub. No.: US 2024/0279750 A1
(43) Pub. Date: Aug. 22, 2024

(54) Diagnostic Primers, Kits and Methods for Viral Detection

(71) Applicant: Genomtec SA, Wroclaw (PL)

(72) Inventors: Miron TOKARSKI, Brzeg (PL); Izabela PIELKA, Konopiska (PL); Malgorzata MALODOBRA-MAZUR, Wroclaw (PL)

(21) Appl. No.: 17/923,444

(22) PCT Filed: May 5, 2021
(86) PCT No.: PCT/EP2021/061918

§ 371 Date: Nov. 4, 2022

(30) Foreign Application Priority Data
May 5, 2020 (GB) 2006663.5
May 5, 2020 (GB) 2006664.3

(51) Int. Cl. C12Q 1/70

(52) U.S. Cl. CPC C12Q 1/701; C12Q 2600/16

(57) ABSTRACT

Provided is a primer set for detecting the N gene of SARS-COV-2 using a reverse transcription loop-mediated isothermal amplification (RT-LAMP) method, comprising at least a first F3 primer comprising a nucleotide sequence at least 90% identical to SEQ ID NO: 1, at least a first B3 primer comprising a nucleotide sequence at least 90% identical to SEQ ID NO: 2, at least a first F2 primer comprising a nucleotide sequence at least 90% identical to SEQ ID NO: 3, at least a first B2 primer comprising a nucleotide sequence at least 90% identical to SEQ ID NO: 4, at least a first F1c primer comprising a nucleotide sequence at least 90% identical to SEQ ID NO: 5 or to SEQ ID NO: 8, at least a first B1c primer comprising a nucleotide sequence at least 90% identical to SEQ ID NO: 6. Also provided are kits and methods for using the described primers, and a primer set for similarly detecting the S gene of SARS-COV-2.

### CROSS-REFERENCE TO RELATED APPLICATIONS

This application is a § 371 National Stage Application of International Application No. PCT/EP2021/061918, filed May 5, 2021, which claims priority to United Kingdom Application No. GB 2006663.5, filed May 5, 2020 and United Kingdom Application No. GB 2006664.3, filed May 5, 2020, the entire contents of which are incorporated herein by reference.

### SEQUENCE LISTING

A computer-readable form (CRF) sequence listing having file name GMT0001PA.txt, created on May 30, 2023 (12,641 bytes), is incorporated herein by reference. The sequences listed in the accompanying sequence listing are shown using standard abbreviations as defined in 37 C.F.R. 1.822.

### FIELD OF INVENTION

The present invention is concerned with diagnostic primers for viral detection, as well as kits, methods and uses associated with such primers. In particular, the present invention relates to sets of primers for the detection of Severe acute respiratory syndrome coronavirus 2; methods for its detection; and the utilization of such primer sets. The invention is intended to be useful in medical diagnostics.

### BACKGROUND OF THE INVENTION

Severe acute respiratory syndrome coronavirus 2 belongs to broad family of viruses—coronaviruses (Coronaviridae). This virus is a positive-sense single-stranded RNA (+ssRNA). Severe acute respiratory syndrome coronavirus 2 (hereafter SARS-COV-2) is a member of the subgenus Sarbecovirus (beta-CoV lineage B). It is unique among known betacoronaviruses in its incorporation of a polybasic cleavage site, a characteristic known to increase pathogenicity and transmissibility in other viruses.

SARS-COV-2 is responsible for infectious, respiratory disease-coronavirus disease 2019 (COVID-19). This has spread globally during a matter of months, causing the 2019-2020 pandemic. Common symptoms include fever, cough, shortness of breath, but in severe cases the disease causes viral pneumonia and multi-organ failure. Currently, there is no vaccine, or any specific antiviral treatment for COVID-19. The standard method of diagnosis is Real-Time reverse transcription polymerase chain reaction (RT-PCR). The combination of chest CT scan, risk factors and symptoms also allows diagnosis. The standard method is time-consuming, while methods other than genetic identification lack the sensitivity and specificity to provide accurate diagnosis.

Therefore, there is a need to develop new, faster, more sensitive and/or more specific methods of SARS-COV-2 diagnosis, which will significantly improve the current rate of diagnosis. Molecular methods based on the detection of specific fragments of the SARS-COV-2 genome are among the most sensitive and specific methods for the pathogen diagnosis. The ideal solution would be a test developed for the needs of basic health care units, at the primary care where patients enter the diagnostic pathway, enabling rapid diagnosis (for example, in less than 20 minutes) and consequently targeted therapy prescription.

The Loop-mediated isothermal amplification (LAMP) method is a single-tube technique for the amplification of DNA/RNA and has been described, for example, in WO0028082A1 and WO0224902A1. A reverse transcription loop-mediated isothermal amplification method (RT-LAMP) has similarly been described, for example, in WO0177317A1, CN103146847B, U.S. Pat. No. 8,389,221B2, and CN102286637B.

The above-mentioned documents do not describe methods for the detection of Severe acute respiratory syndrome coronavirus 2.

Therefore, the need to provide a set of primers enabling the POC diagnostic method for SARS-CoV-2 (by the RT-LAMP method of nucleic acid amplification) intended for laboratory-independent healthcare facilities use still very much exists. The proposed RT-LAMP-based diagnostic primers allow identification of the SARS-COV-2 virus with a very low limit of detection (≥50 GE/μl) in a short time (≤20 min). The proposed set of primers for intellectual protection solves the earlier discussed problem of real-time quantification obtained for low copies of the pathogen's RNA in rapid time.

### SUMMARY OF THE INVENTION

In a first aspect, there is provided a primer set for detecting SARS-COV-2 using a reverse transcription loop-mediated isothermal amplification (RT-LAMP) method, comprising:
i) at least a first F3 primer comprising a nucleotide sequence at least 90% identical to SEQ ID NO: 1;
ii) at least a first B3 primer comprising a nucleotide sequence at least 90% identical to SEQ ID NO: 2;
iii) at least a first F2 primer comprising a nucleotide sequence at least 90% identical to SEQ ID NO: 3;
iv) at least a first B2 primer comprising a nucleotide sequence at least 90% identical to SEQ ID NO: 4;
v) at least a first F1c primer comprising a nucleotide sequence at least 90% identical to SEQ ID NO: 5 or to SEQ ID NO: 8; and
vi) at least a first B1c primer comprising a nucleotide sequence at least 90% identical to SEQ ID NO: 6.

In a still further aspect, there is provided a kit for detecting Severe acute respiratory syndrome coronavirus 2 (SARS-COV-2) in a sample, the kit comprising reagents for amplifying RNA in a sample using a RT-LAMP technique and a primer set according to any of the above aspects.

In a yet further aspect, there is provided a method of detecting SARS-COV-2 in a sample, comprising amplifying by isothermal amplification at least a portion of the SARS-COV-2 genome using a primer set according to any of the above aspects.

The first subject of the invention is a set of primers for amplifying the nucleotide sequence of the N gene of the SARS-COV-2 virus (SEQ ID NO: 14). The set is characterized by comprising the internal primers with the following nucleotide sequences a) and b), as well as the set of external primers comprising the following nucleotide sequences c) and d) and being specific for selected region of the Severe acute respiratory syndrome coronavirus 2 N gene:
a) 5′ TCSYYTACTGCTGCCTGGAG 3′-(the nucleotide sequence SEQ ID NO: 5 or the sequence created by single nucleotide changes, substitution or deletion of a single nucleotide connected or not connected by TTTT bridge to the sequence 5′ CGTTCCTCATCACGTAGTCG 3′-(the nucleotide sequence SEQ ID NO: 3 or the sequence created by single nucleotide changes, substitution or deletion of a single nucleotide);
b) 5′ TCTCCTGCTAGAATGGCTGGCA 3′-(the nucleotide sequence SEQ ID NO: 6 or the sequence created by single nucleotide changes, substitution or deletion of a single nucleotide) connected or not connected by TTTT bridge to the sequence 5′ TCTGTCAAGCAGCAGCAAAG 3′-(the nucleotide sequence SEQ ID NO: 4 or the sequence created by single nucleotide changes, substitution or deletion of a single nucleotide);
c) 5′ CGGCAGTCAAGCCTCTTC 3′ the nucleotide sequence SEQ ID NO: 1 or the sequence created by single nucleotide changes, substitution or deletion of a single nucleotide and
d) 5′ TTGCTCTCAAGCTGGTTCAA 3′ the nucleotide sequence SEQ ID NO: 2 or the sequence created by single nucleotide changes, substitution or deletion of a single nucleotide

Complementary with the above nucleotide sequence, the invention comprises below loop primer sequence comprising the Severe acute respiratory syndrome coronavirus 2 N gene SEQ ID NO: 7:-5′ ATGGCGGTGATGCTGCTCT 3′ or sequence resulting from single nucleotide exchanges, substitution or deletion of a single nucleotide.

The second subject of the invention is a method for Severe acute respiratory syndrome coronavirus 2 detection, characterized by amplification of a selected region of the SARS-COV-2 genome (that is, a fragment of the N gene) using the set of primers characterized in the first subject of the invention, wherein the method of detection employed is RT-LAMP (reverse transcription LAMP).

In a preferred embodiment, the nucleic acid amplification is carried out at 64° C.-70° C., in particular 68° C., for 40 min.

In another preferred embodiment of the invention, the end-point type reaction is carried out at the condition mention above, and ended at 80° C., 5 min.

The third subject of the invention is a method for the detection of SARS-COV-2 infection, wherein the method uses RT-LAMP based nucleic acid amplification with a specific set of primers (first subject of the invention), detecting and amplifying a specific region of the SARS-COV-2 genome (described in the second subject of the invention).

The fourth subject of the invention is the reaction mix used for detection of infection caused by SARS-COV-2, comprising the set of primers described in the first subject of the present invention.

In a preferred embodiment of the invention, the reaction mix for detection of infection caused by SARS-COV-2 comprises 10 μl WarmStart LAMP KIT (RNA&DNA) (NEB).

In another preferred embodiment of the invention, the individual amplification primers as defined in the first object of the invention, wherein the primers have the following concentrations: 0.13 μM F3 (SEQ ID NO: 1), 0.13 μM B3 (SEQ ID NO: 2), 1.0 μM FIP (SEQ ID NO: 5 or 8 and 3), 1.0 μM BIP (SEQ ID NO: 6 and 4), 0.25 μM LoopB (SEQ ID NO: 7).

Additionally, the other components of the reaction mix include (at given concentrations): double-stranded DNA fluorescent marker-EvaGreen≤1× or Fluorescent Dye≤0,4 ul or Green Fluorescent Dye≤0,8 ul or Syto-13≤16 μM or SYTO-82≤16 μM or other double-stranded DNA fluorescent dye at a concentration not inhibiting the amplification reaction.

### BRIEF DESCRIPTION OF THE FIGURES

FIG. 1 shows the sequence of the N gene of SARS-COV-2, as well as the alignment of the primers discussed herein for amplification of that sequence.

FIG. 2 shows the first step for virus detection, cDNA synthesis by reverse transcriptase.

FIG. 3 shows the amplification steps of the RT-LAMP procedure.

FIGS. 4 and 5 show the sensitivity characteristics of the LAMP method of amplifying the N gene of SARS-COV-2 by gel electrophoresis and by a trace of the real-time amplification of a series of standard dilutions.

FIG. 6 illustrates the specificity of the method of the invention measured by setting a LAMP-based reaction (end-point) with DNA standards of numerous pathogens.

FIG. 7 shows the alignment of multiple N sequences from several clinical samples.

FIG. 8 shows the sequence of the S gene of SARS-COV-2, as well as the alignment of the primers discussed herein for amplification of that sequence.

FIGS. 9 and 10 show the sensitivity characteristics of the LAMP method of amplifying the S gene of SARS-COV-2 by gel electrophoresis and by a trace of the real-time amplification of a series of standard dilutions.

### DETAILED DESCRIPTION

The advantage of the proposed set of primer as stated in the invention for the detection of SARS-CoV-2, as well as the method of detection of SARS-COV-2 infection and the method of detection of amplification products specific to the SARS-COV-2 genome, is its ability to be used in POC laboratory-independent medical diagnostic setting, detecting specific pathogen on a portable genetic analyzer (subject of another intellectual property filling) with an extremely high sensitivity and specificity. In turn, the use of fluorescent dye to detect the amplified genetic product(s) increases the sensitivity of the method, allows the detection limit to be reduced (to 50 genome equivalents (GE)/μl), and enables quantitative measurement of the SARS COV-2 in the test sample.

### N Gene Detection

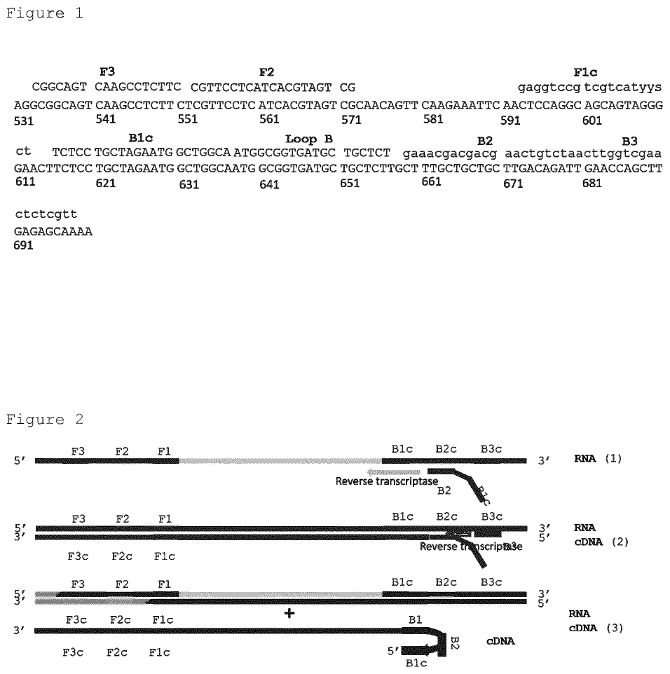

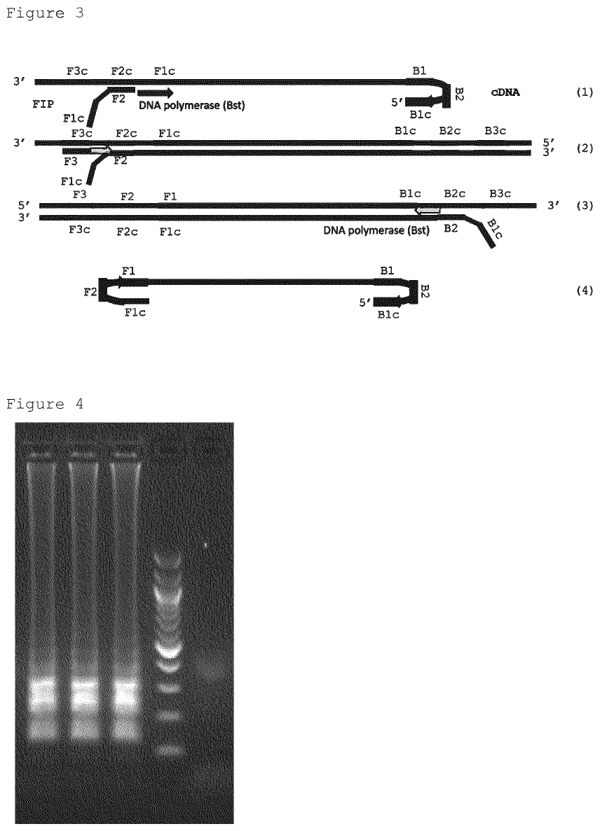

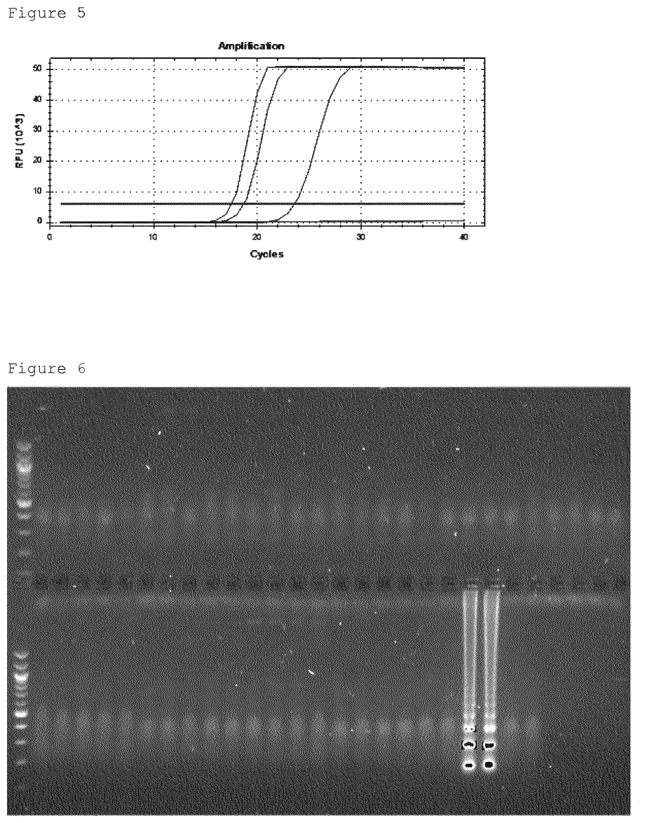

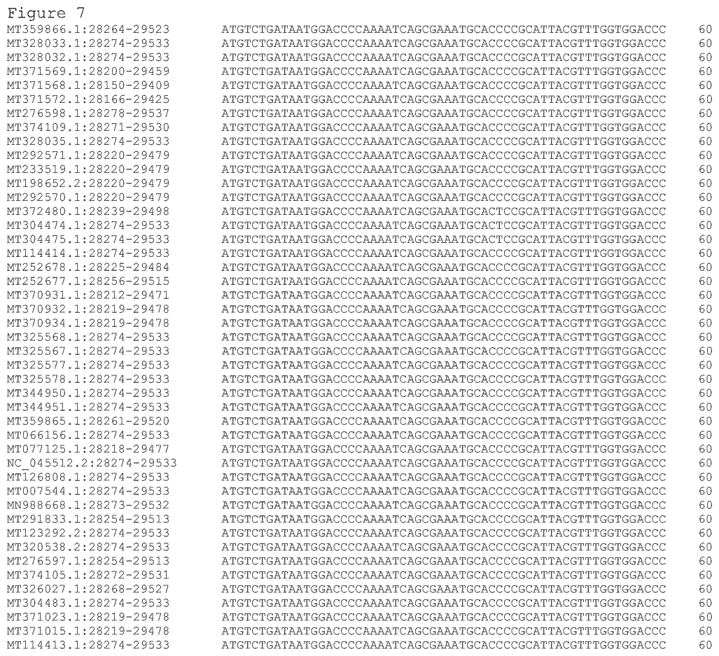

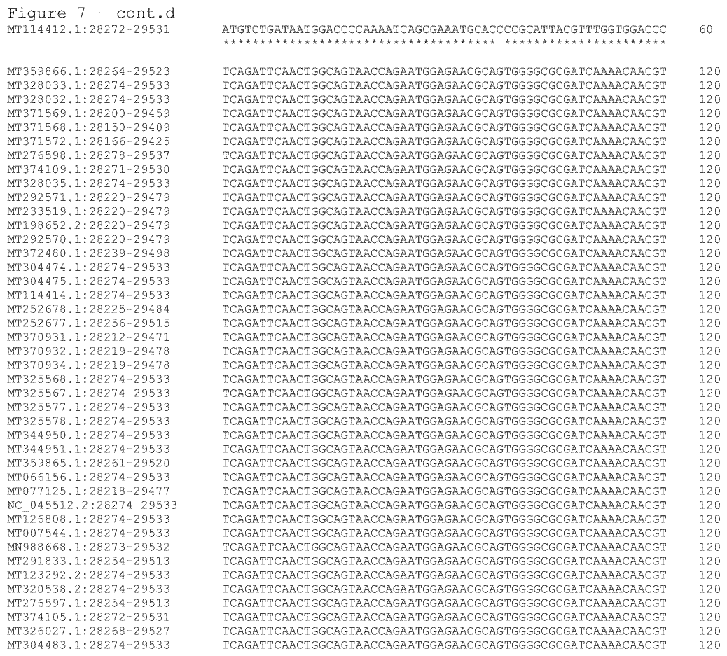

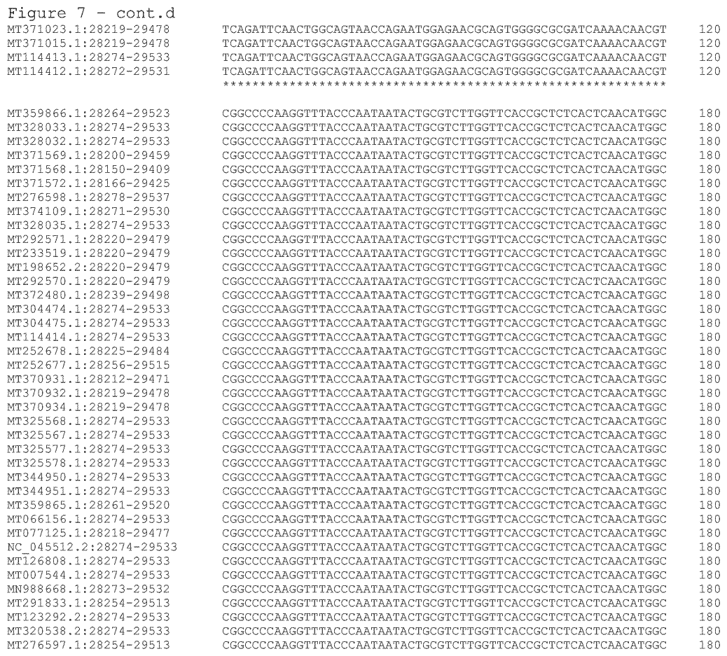

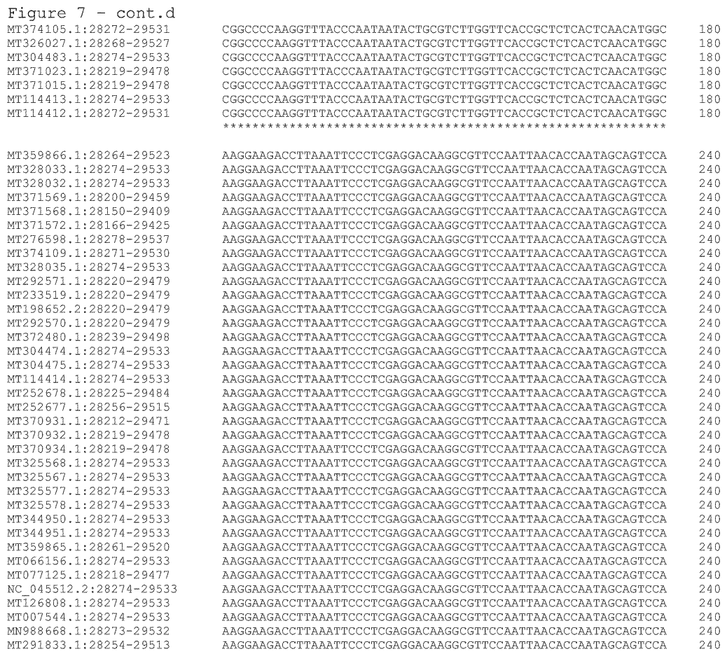

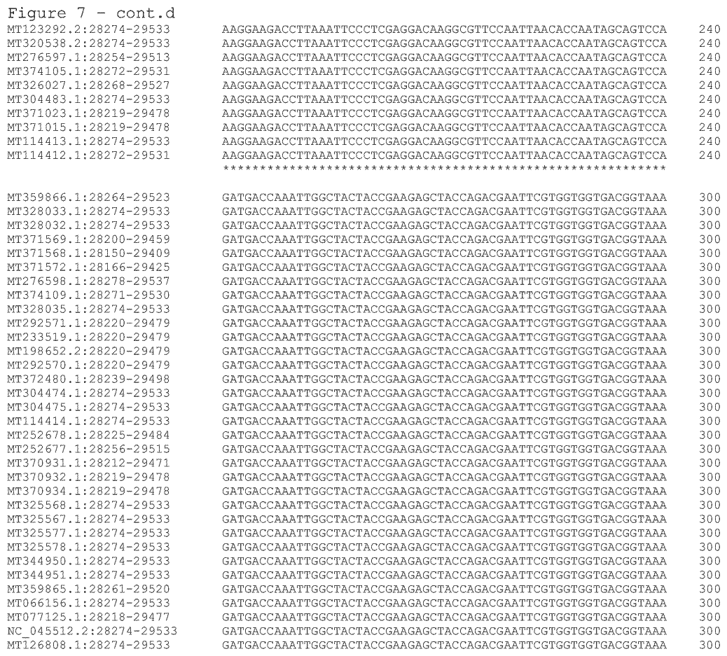

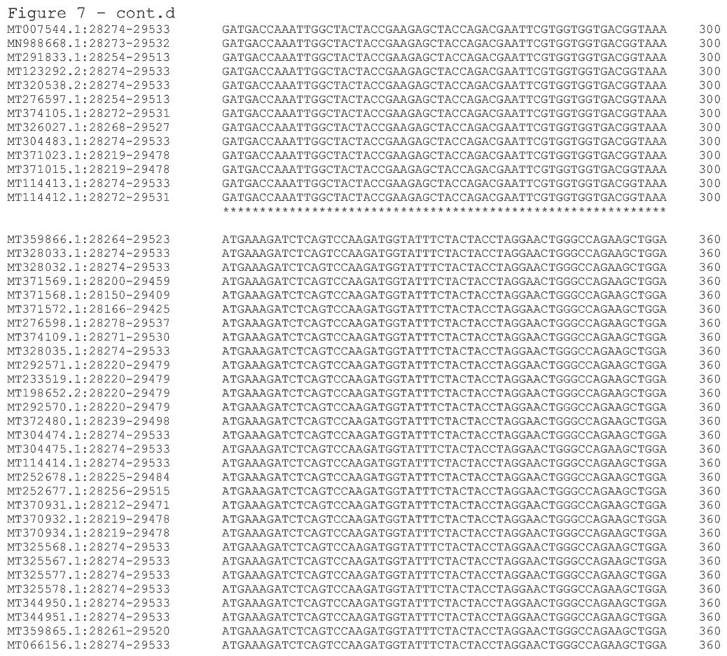

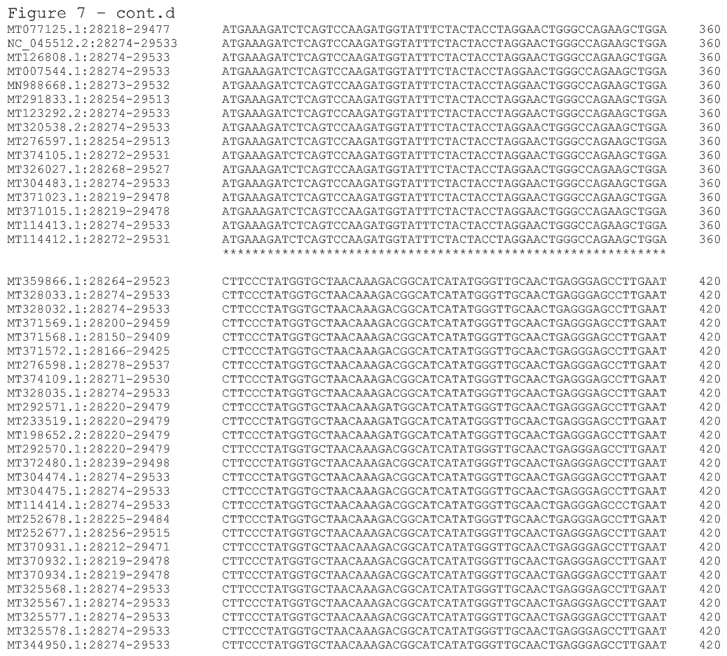

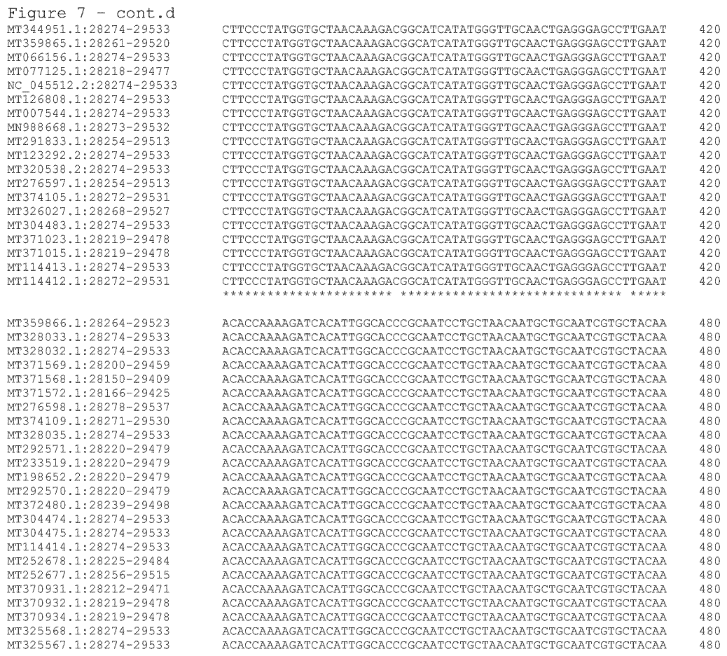

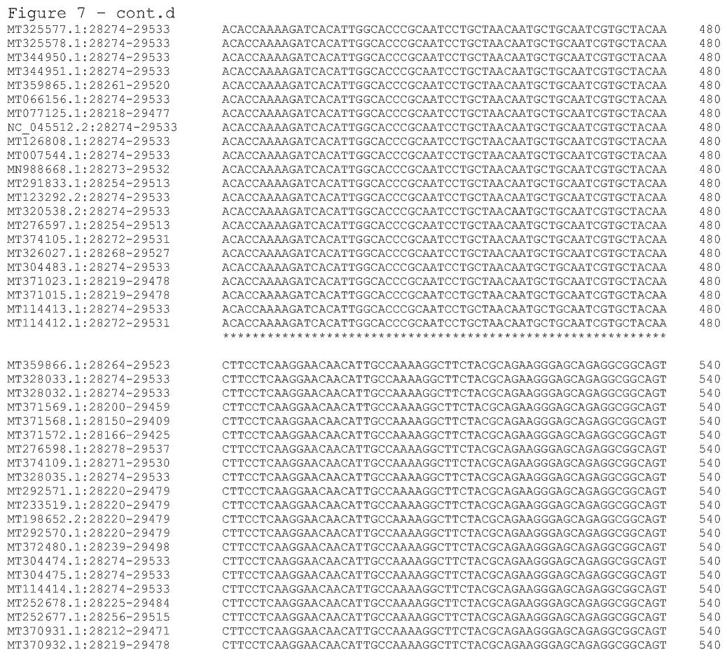

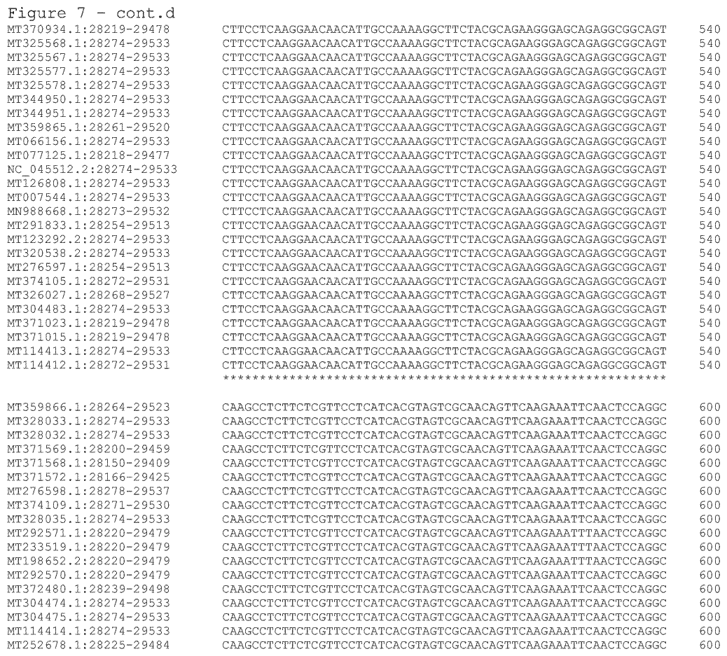

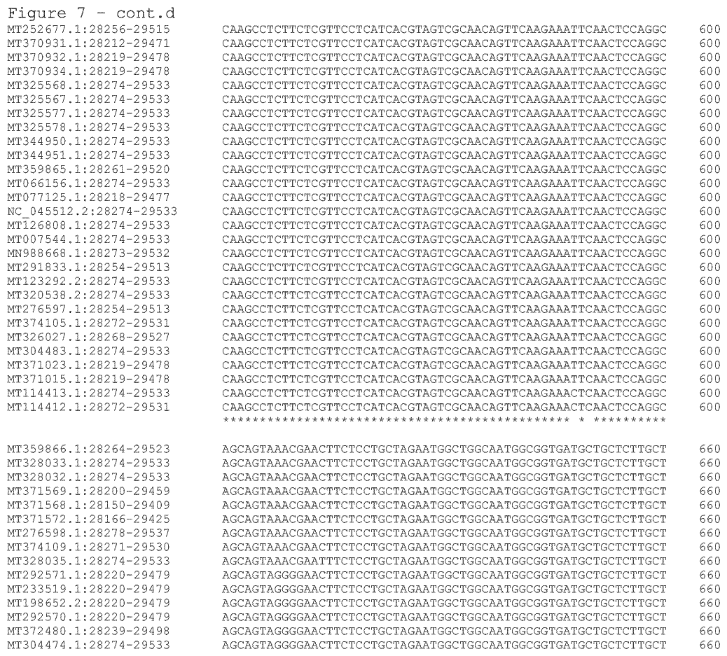

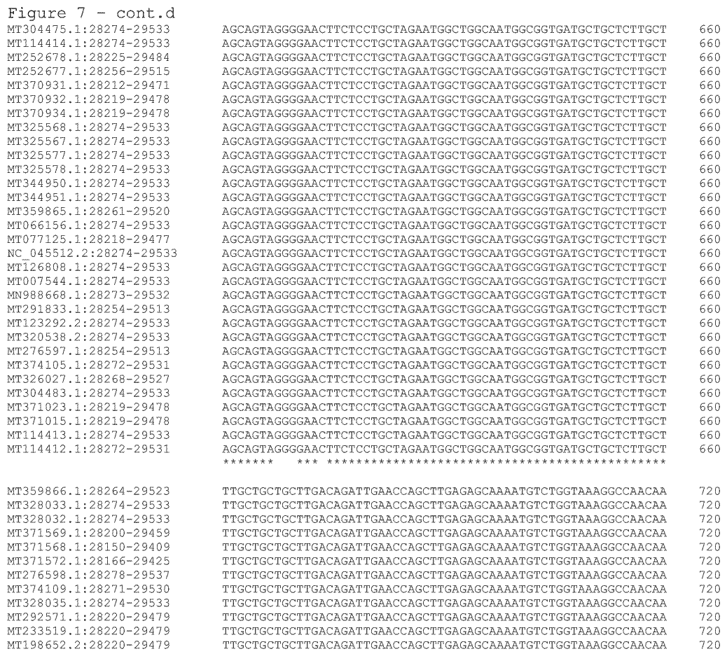

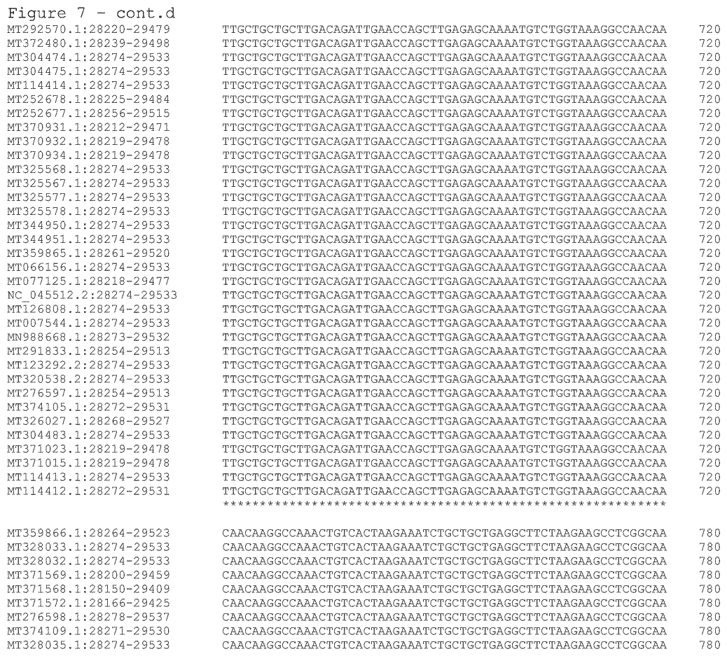

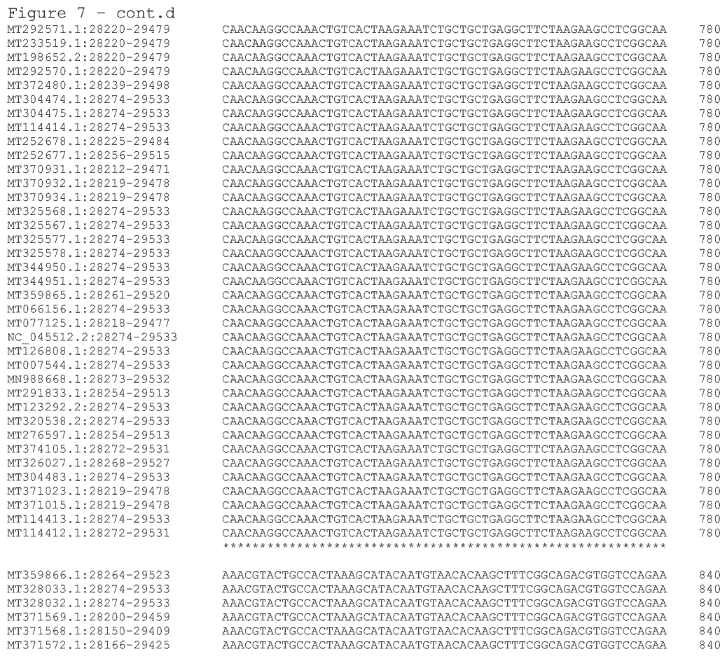

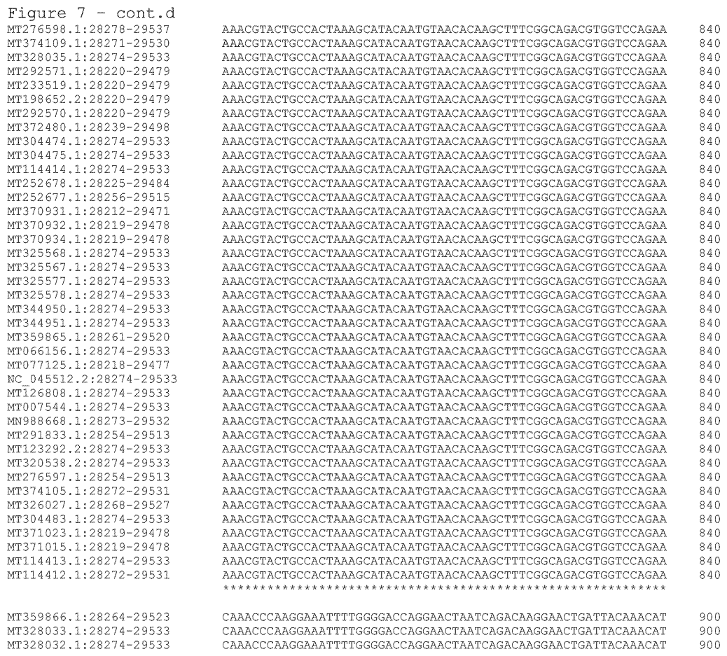

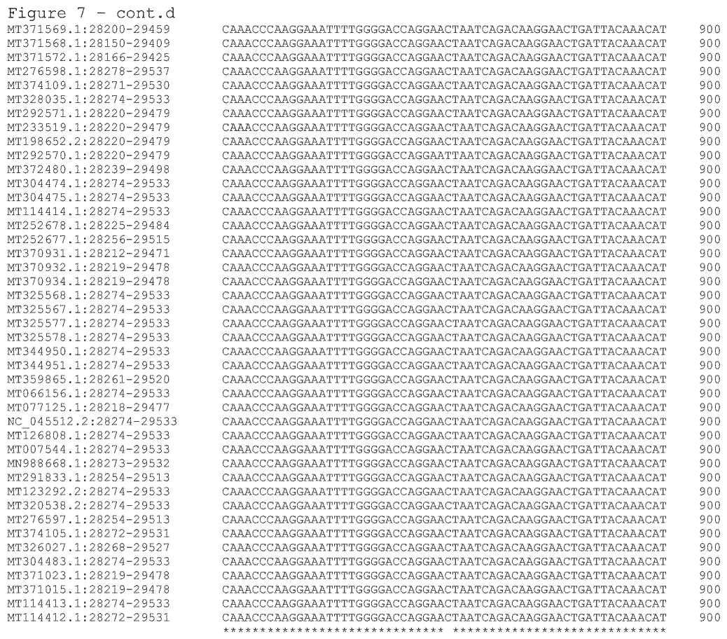

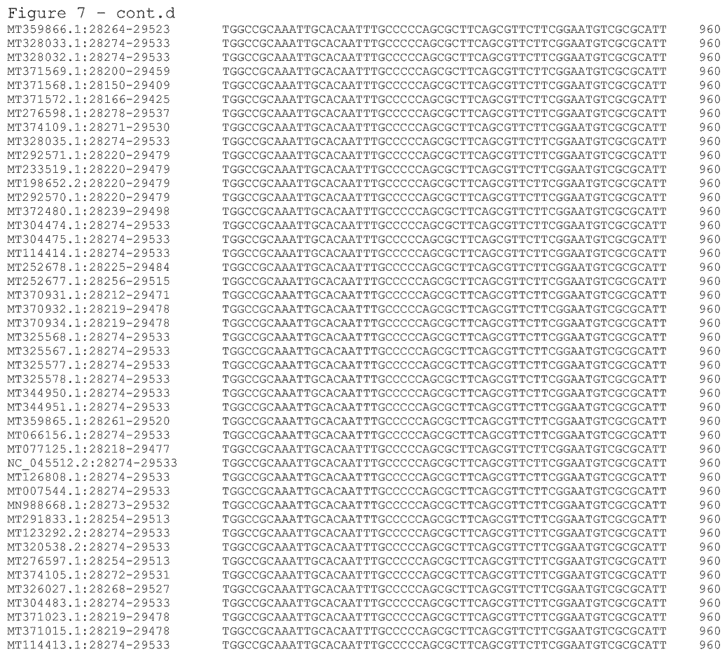

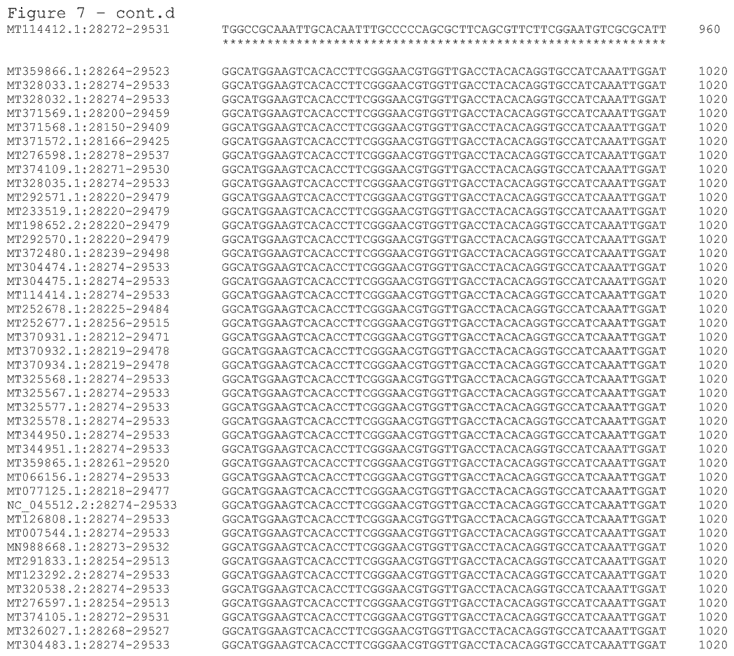

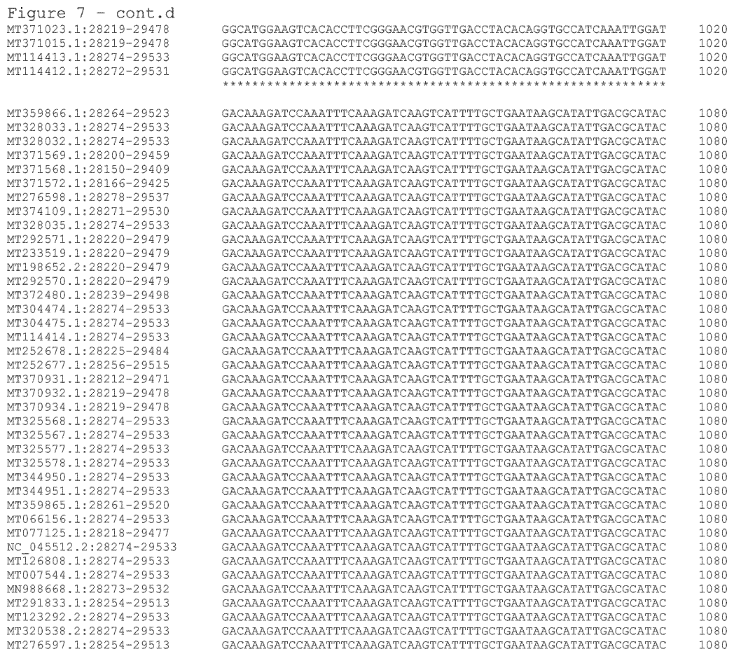

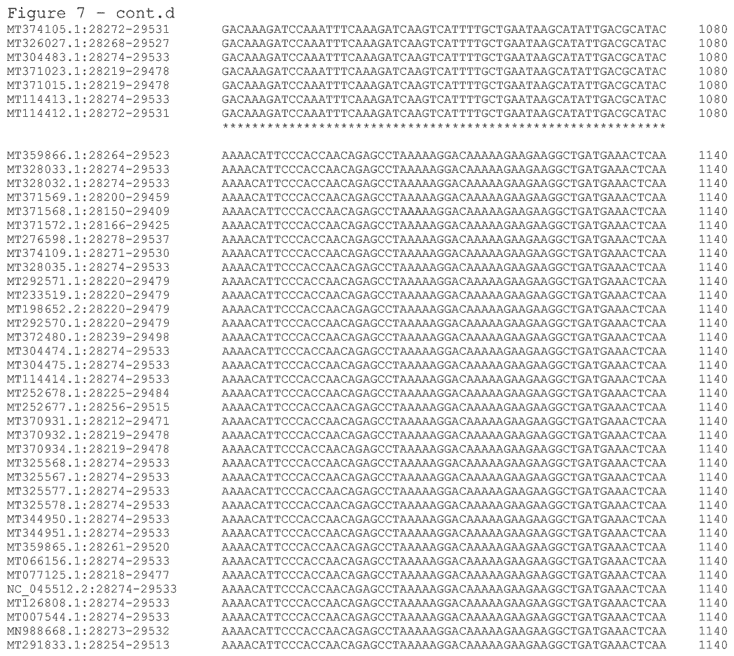

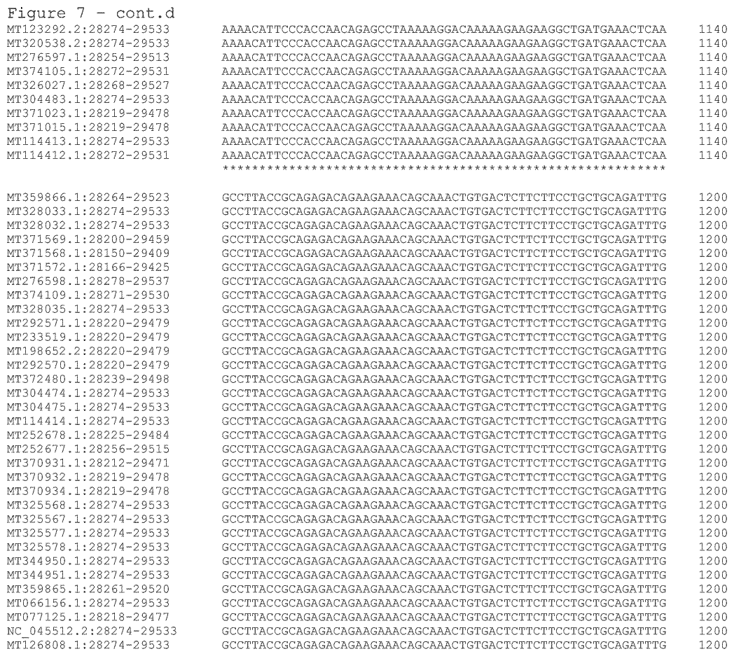

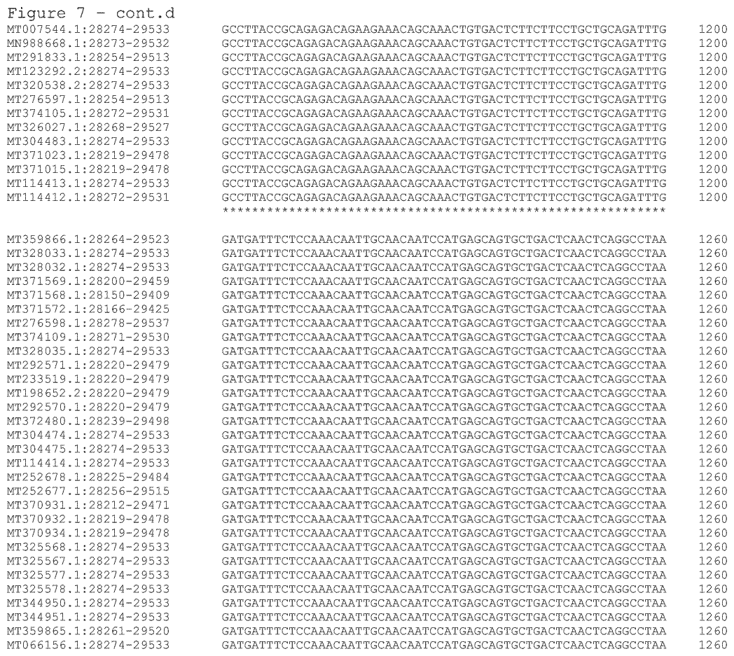

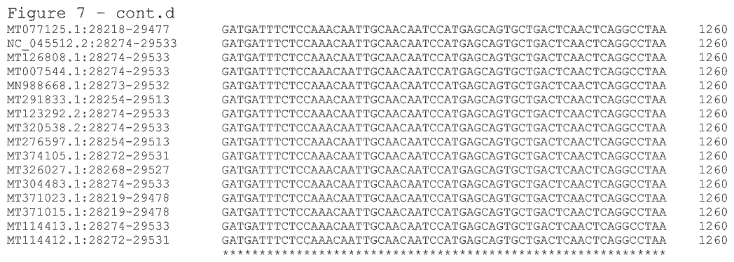

For the SARS-COV-2 detection using RT-LAMP, five primers are used that are specific to detect a target sequence/region of the pathogen's nucleic acid sequence (the N gene of SARS-COV-2, FIG. 1 and SEQ ID NO: 14; see also the alignment of multiple N sequences from several clinical samples in FIG. 7). The first step for virus detection is cDNA synthesis by reverse transcriptase (FIG. 2). The process is initialized by B2 primer that binds to the specific region of the N gene, and the reverse transcriptase continues the synthesis of the cDNA (1). Next, the outer B3 primer binds to the outside region of the BIP primer (2), and the new cDNA is synthesized by reverse transcriptase, concurrently releasing the single-stranded cDNA from the previous step (3). The amplification (FIG. 3) is initiated by one of the inner primers (F2) that creates with F1c the FIP primer (1). After the initialization step, the strand-displacement DNA polymerase (especially Bst polymerase) extends the FIP primer. This step ends with a synthesis of the first LAMP product, which is then displaced by the synthesis of the second product initiated by one of the outer primers F3 (2). The strand synthesized from the F3 primer together with the cDNA from the initial step of the process forms double stranded DNA (3). The product displaced by F3 primer contains complementary sequences on both ends which enable self-annealing and forming dumbbell-like structure, which is then subject to LAMP cycling amplification (4). As the amplification proceeds, the products that are synthesized creates concatemer structures that serve for even more sites for amplification initiations. The result of multiple site initiation is the accumulation of double-stranded DNA of different length and structure (so-called cauliflower structures) that can be next detected using intercalating fluorescent dyes as the real-time approach.

It is not anticipated that sequences exactly identical or exactly complementary to those recited in the sequences disclosed herein must necessarily be used for the invention to be effective. For example, the primers used can comprise sequences which are at least 90%, at least 95%, at least 98%, at least 99% or 100% identical to the sequences disclosed herein, and/or which differ from those sequences as a result of single nucleotide exchanges, substitution or deletion of a single nucleotide. One or more of the primers can consist of the sequences disclosed herein. IUPAC sequence convention is used, such that, for example, ‘S’ stands for ‘strong’ bases (C, G) and ‘Y’ stands for pyrimidine bases (C, T). FIG. 7 illustrates multiple sequence alignment of SARS-COV-2 N gene from several clinical samples.

The F1c and F2 primers are generally connected to form one double primer, known as the Forward Inner Primer (FIP). Likewise, B1c and B2 primers are generally connected to form one double primer, known as the Backward Inner Primer (BIP). These primers may be directly connected, or may be connected by a nucleotide bridge. Such a bridge may be between one and ten nucleotides in length. In particular, it has been observed that certain sequences, such as a TTTT sequence, can help in the formation of loop structures. Accordingly, in some embodiment the bridge within the FIP and/or BIP double primers may be a TTTT nucleotide sequence.

Exemplary implementations of the invention and supporting data are shown in the drawings, in which FIG. 4 shows the sensitivity characteristics of the LAMP method, where a specific signal was obtained with the RNA standard (Twist Synthetic SARS-COV-2 RNA Control 1; Twist Bioscience) in the range of 1000-50 copies/μl, while the NTC reaction was negative; FIG. 1: Line 1: 50 SARS-CoV-2 copies; line 2: 100 SARS-COV-2 copies; line 3: 1000 SARS-COV-2 copies; line 4: DNA molecular-weight size marker (Quick-Load® Purple 100 bp DNA Ladder, NewEngland Biolabs); line 5: NTC.

FIG. 5 illustrates the sensitivity of the LAMP method of the invention measured by tracking a trace of the real-time amplification of a series of the standard dilutions (Twist Synthetic SARS-COV-2 RNA Control 1 (Twist Bioscience) in the range of 1,000-50 copies/μl of the DNA standard tested.

The results of SARS-COV-2 detected in real-time are presented in Table 1, giving indication of the minimum time necessary to detect the fluorescence signal.

FIG. 6 illustrates the specificity of the method of the invention measured by setting a LAMP-based reaction (end-point) with DNA standards of numerous pathogens, often present in biological material as the physiological pathogens, pathogens which may appear as the result of co-infection, or which have similar genomic sequences.

Line 1: DNA molecular-weight marker (Quick-Load® Purple 100 bp DNA Ladder, NewEngland Biolabs); Lines 2 and 3: Mycoplasma genitalium; Lines 4 and 5: Streptococcus pyogenes; Lines 6 and 7: Enterococcus faecalis; Lines 8 and 9: Moraxella catarrhalis; Lines 10 and 11: Legionella pneumophila; Lines 12 and 13: Escherichia coli; Lines 14 and 15: Candida albicans; Lines 16 and 17: Mycoplasma pneumoniae; Lines 18 and 19: Klebsiella pneumoniae; Lines 20 and 21: Staphylococcus aureus methicilin sensitive (MSSA); Lines 22 and 23: Enterococcus faecium; Lines 24 and 25: Acinetobacter baumannii; Lines 26 and 27: Mycoplasma hominis; Line 28 and 29: Ureoplasma urealyticum; Lines 30 and 31: Haemophilus influenzae; Lines 32 and 33: Human genomic DNA; Lines 34 and 35: Bordetella pertussis; Lines 36 and 37: Staphylococcus aureus methicilin resistant (MRSA); Lines 38 and 39: Pseudomonas aeruginosa; Lines 40 and 41: Listeria monocytogenes; Line 42 and 43: Haemophilus ducreyi; Lines 44 and 45: Campylobacter jejuni; Lines 46 and 47: Chlamydophila pneumoniae; Lines 48 and 49: Twist Synthetic SARS-COV-2 RNA Control 2 (Twist Bioscience); Lines 50 and 51: NTC (no-template control).

### S Gene Detection

In addition to the sequences and methods discussed above to amplify the N gene of the SARS-CoV-2 virus, there is also provided herewith a set of primers for amplifying the nucleotide sequence of the S gene of the SARS-COV-2 virus (SEQ ID NO: 27). The set is characterized by comprising the internal primers with the following nucleotide sequences a) and b), as well as the set of external primers comprising the following nucleotide sequences c) and d) and being specific for selected region of the Severe acute respiratory syndrome coronavirus 2 S gene:
a) 5′ CATGGAACCAAGTAACATTGGAAAA 3′-(the nucleotide sequence SEQ ID NO: 19 or the sequence created by single nucleotide changes, substitution or deletion of a single nucleotide connected or not connected by TTTT bridge to the sequence 5′ TTTTCAGATCCTCAGTTTTACATTC 3′-(the nucleotide sequence SEQ ID NO: 17 or the sequence created by single nucleotide changes, substitution or deletion of a single nucleotide);
b) 5′ CTCTGGGACCAATGGTACTAAGAG 3′-(the nucleotide sequence SEQ ID NO: 20 or the sequence created by single nucleotide changes, substitution or deletion of a single nucleotide) connected or not connected by TTTT bridge to the sequence 5′ GACTTCTCAGTGGAAGCA 3′-(the nucleotide sequence SEQ ID NO: 18 or the sequence created by single nucleotide changes, substitution or deletion of a single nucleotide);
c) 5′ GGTGTTTATTACCCTGACAAAG 3′ the nucleotide sequence SEQ ID NO: 15 or the sequence created by single nucleotide changes, substitution or deletion of a single nucleotide and
d) 5′ GTACCAAAAATCCAGCCTC 3′ the nucleotide sequence SEQ ID NO: 16 or the sequence created by single nucleotide changes, substitution or deletion of a single nucleotide

Complementary with the above nucleotide sequence, the set of primers can further comprise the below loop primer sequences comprising sequence complementary to the Severe acute respiratory syndrome coronavirus 2 S gene SEQ ID NO: 21:-5′ GAAAGGTAAGAACAAGTCCTGAGT 3′ and sequence identical to the Severe acute respiratory syndrome coronavirus 2 S gene SEQ ID NO: 22: 5 CTGTCCTACCATTTAATGATGGTGT 3′ or sequences resulting from single nucleotide exchanges, substitution or deletion of a single nucleotide.

Aside from the use of different primers as discussed, and the presence of an additional loop primer sequence (that is, SEQ ID NO: 21), this set of primers for amplifying the nucleotide sequence of the S gene of the SARS-COV-2 virus can be used in the same way as described above for the primers corresponding to the N gene, and/or as further described below.

Accordingly, further provided is a method for Severe acute respiratory syndrome coronavirus 2 detection, characterized by amplification of a selected region of the SARS-COV-2 genome (that is, a fragment of the S gene) using the set of primers characterized above, wherein the method of detection employed is RT-LAMP (reverse transcription LAMP).

In a preferred embodiment, the nucleic acid amplification is carried out at 60° C., for 40 min.

In another preferred embodiment of the invention, the end-point type reaction is carried out at the condition mention above, and ended at 80° C., 5 min.

### EXAMPLES

### Example 1-N Gene-Primer Sequences

The specific sequences of oligonucleotides used to detect Severe acute respiratory syndrome coronavirus 2 genetic material (the N gene) utilizing LAMP technology are shown on FIG. 1, FIG. 2 and FIG. 3 and characterized below:
1. Oligonucleotide sequence SARS-COV-2 NF3: 5′ CGGCAGTCAAGCCTCTTC 3′ (SEQ ID NO: 1) is a sequence identical to the Severe acute respiratory syndrome coronavirus 2 N gene (strand 5′-3′), which from the 3′ end is adjacent to the F2 primer.
2. Oligonucleotide sequence SARS-COV-2 NB3: 5′ TTGCTCTCAAGCTGGTTCAA 3′ (SEQ ID NO: 2) is a complementary fragment of Severe acute respiratory syndrome coronavirus 2 N gene (strand 5′-3′) separated by 127 nucleotides from the 3′ end of oligonucleotide 1.
3. Oligonucleotide sequence SARS-COV-2 NF2: 5′ CGTTCCTCATCACGTAGTCG 3′ (SEQ ID NO: 3) is a sequence identical to the Severe acute respiratory syndrome coronavirus 2 N gene (strand 5′-3′) sequence separated by 1 nucleotide from the 3 ‘end of oligonucleotide 1.
4. Oligonucleotide sequence SARS-COV-2 NB2: 5’ TCTGTCAAGCAGCAGCAAAG 3′ (SEQ ID NO: 4) is a complementary fragment of Severe acute respiratory syndrome coronavirus 2 N gene (strand 5′-3′) separated by 107 nucleotides from the 3′ end of oligonucleotide 1.
5. Oligonucleotide sequence SARS-COV-2 NF1c: 5′ TCSYYTACTGCTGCCTGGAG 3′ (SEQ ID NO: 5) is a complementary fragment of Severe acute respiratory syndrome coronavirus 2 N gene (strand 5′-3′) separated by 41 nucleotides from the 3′ end of oligonucleotide 1. In a particular embodiment, the sequence SARS-COV-2 NF1′: 5′ TCCCCTACTGCTGCCTGGAG 3′ (SEQ ID NO: 8) can be used.
6. Oligonucleotide sequence SARS-COV-2 NB1c: 5′ TCTCCTGCTAGAATGGCTGGCA 3′ (SEQ ID NO: 6) is a sequence identical to the Severe acute respiratory syndrome coronavirus 2 N gene separated by 64 nucleotides from the 3′ end of oligonucleotide 1.
7. Oligonucleotide sequence SARS-COV-2 NLoopB: 5′ ATGGCGGTGATGCTGCTCT 3′ (SEQ ID NO: 7).

The F1c and F2 oligonucleotide sequences are preferably linked by a TTTT bridge and used in the form of a Forward Inner Primer (FIP); for example: 5′ TCSYYTACTGCTGCCTGGAGNNNNCGTTCCTCATCACGTAGTCG 3′ (SEQ ID NO: 9); 5′ TCSYYTACTGCTGCCTGGAGTTTTCGTTCCTCATCACGTAGTCG 3′ (SEQ ID NO: 10); or most particularly 5′ TCCCCTACTGCTGCCTGGAGTTTTCGTTCCTCATCACGTAGTCG 3′ (SEQ ID NO: 11)—where N is any nucleotide.

The B1c and B2 oligonucleotide sequences are preferably linked via a TTTT bridge and are used in the form of a Backward Inner Primer (BIP); for example: 5′ TCTCCTGCTAGAATGGCTGGCANNNNTCTGTCAAGCAGCAGCAAAG 3′ (SEQ ID NO: 12); or most particularly 5′ TCTCCTGCTAGAATGGCTGGCATTTTTCTGTCAAGCAGCAGCAAAG 3′ (SEQ ID NO: 13)—where N is any nucleotide.

### Example 2

Severe acute respiratory syndrome coronavirus 2 N gene amplification method utilizing LAMP technology with oligonucleotides characterized in Example 1, with the following composition of the reaction mixture:
10,0 μl WarmStart LAMP KIT (RNA&DNA)
0,13 μM F3
0,13 μM B3
1,00 μM FIP
1,00 μM BIP
0,25 μM LoopB
   Fluorescent Dye Interacting with Double-Stranded DNA:
EvaGreen≤1× or Fluorescent dye 50× (New England Biolabs) at 0,4 μl, or Green Fluorescent Dye (Lucigen) at ≤0,8 μl, or
Syto-13≤16 μM or SYTO-82≤16 μM, or other fluorescent dye interacting with double-stranded DNA at a concentration that does not inhibit the amplification reaction.
   DNA template≥50 copies/reaction

### Example 3

Severe acute respiratory syndrome coronavirus 2 N gene amplification method using oligonucleotides characterized in Example 1 and Example 2 in LAMP technology with the composition of the reaction mixture characterized in Example 3 with the following temperature profile:
1) 68° C., 40 min
2) preferably at 80° C. end-point reactions, 5 min. Such end point reactions aim to denature the polymerase in order to stop amplification.

### Example 4

The method of amplification and detection of the Severe acute respiratory syndrome coronavirus 2 N gene using oligonucleotides characterized in Example 1 in LAMP technology with the composition of the reaction mixture characterized in Example 2 with the temperature profile characterized in Example 3 and the detection method described below.

Fluorescent dye has the ability to interact with double-stranded DNA present in the reaction mixture in the amount of 1 μL EvaGreen 20X-0,8 μl or concentration≤1×; ≤16 μM for Green Fluorescent Dye (Lucigen), SYTO-13 and SYTO-82 respectively; measurement affected include the Real-Time and/or end-point reactions. Excitation wavelength in the range similar to FAM dye include: 490-500 nm (optimally 494 nm) for EvaGreen dyes, Fluorescent dye 50× (New England Biolabs), Green Fluorescent Dye (Lucigen); 535 nm (optimally 541 nm) for SYTO-13 and SYTO-82. Emission wavelength in the range 509-530 nm (optimally 518 nm) for EvaGreen dyes and Green Fluorescent Dye (Lucigen); 556 nm (optimally 560 nm) for SYTO-13 and SYTO-82-detection method. Changes in detection were recorded 1 minute from the start of the reaction for Severe acute respiratory syndrome coronavirus 2 and negative control.

### Example 5 Method Sensitivity

Sensitivity was determined by setting-up RealTime-RT-LAMP for a series of dilutions of the Twist Synthetic SARS-COV-2 RNA Control 1 (Twist Bioscience); with a minimum amount of 50 copies of viral RNA in the reaction mixture, where product was measured in a real time-FIG. 2 (RealTime-RT-LAMP for a series of dilutions).

The time after which it is possible to detect the emitted fluorescence corresponding to a specific SARS-COV-2 genome fragment for given copy number is shown in Table 1.

Conclusion: the characterized primers enable the detection of Severe acute respiratory syndrome coronavirus 2 by detecting a fragment of the N gene with a minimum of 50 copies/reaction mixture.

TABLE 1
Time needed to detect fluorescence for individual dilutions of
the Severe acute respiratory syndrome coronavirus 2 RNA standard
(Twist Synthetic SARS-CoV-2 RNA Control 2, Twist Bioscience).

| | Time of exceeding the fluorescence |
|---|---|
| Sample | baseline [min] |
| SARS-CoV-2 NTC | Undetermined |
| SARS-CoV-2 50 copies | 23.57 |
| SARS-CoV-2 100 copies | 18.67 |
| SARS-CoV-2 1 000 copies | 17.49 |

The superiority of the amplification method and set of primers (oligonucleotides) characterized in this patent over other tests based on the Real Time-RT-LAMP technology lies in its high sensitivity shown in FIG. 1 and the dramatic reduction of the analysis time shown in FIG. 2 and Table 1 when compared to standard RT-PCR techniques.

Some embodiments are exemplified in the following numbered paragraphs:
1) A set of primers for the amplification of the nucleotide sequence of the N gene of Severe acute respiratory syndrome coronavirus 2, characterized in that it contains a set of internal primers with the following nucleotide sequences a) and b), as well as a set of external primers containing the following nucleotide sequences c) and d):
   a) 5′ TCSYYTACTGCTGCCTGGAG 3′-(the nucleotide sequence SEQ ID NO: 5 or the sequence created by single nucleotide changes, substitution or deletion of a single nucleotide connected or not connected by TTTT bridge to the sequence 5′ CGTTCCTCATCACGTAGTCG 3′-(the nucleotide sequence SEQ ID NO: 3 or the sequence created by single nucleotide changes, substitution or deletion of a single nucleotide);
   b) 5′ TCTCCTGCTAGAATGGCTGGCA 3′-(the nucleotide sequence SEQ ID NO: 6 or the sequence created by single nucleotide changes, substitution or deletion of a single nucleotide) connected or not connected by TTTT bridge to the sequence 5′ TCTGTCAAGCAGCAGCAAAG 3′-(the nucleotide sequence SEQ ID NO: 4 or the sequence created by single nucleotide changes, substitution or deletion of a single nucleotide);
   c) 5′ CGGCAGTCAAGCCTCTTC 3′ the nucleotide sequence SEQ ID NO: 1 or the sequence created by single nucleotide changes, substitution or deletion of a single nucleotide and
   d) 5′ TTGCTCTCAAGCTGGTTCAA 3′ the nucleotide sequence SEQ ID NO: 2 or the sequence created by single nucleotide changes, substitution or deletion of a single nucleotide
2) Set of primers according to clause 1, characterized by the fact that it contains the loop primer of sequence being identical to the Severe acute respiratory syndrome coronavirus 2 N gene SEQ ID NO: 7: 5′ ATGGCGGTGATGCTGCTCT 3′ or sequence resulting from single nucleotide exchanges, substitution or deletion of single nucleotides.
3) A method for detecting Severe acute respiratory syndrome coronavirus 2, characterized in that the selected region of the viral genome nucleic acid sequence is amplified using the primer set as defined in clause 1 and clause 2, wherein the amplification method is the RT-LAMP method.
4) Virus detection method according to clauses. 3. The process described in clause 3, wherein the nucleic acid amplification is carried out at a temperature profile: −68° C., 40 min
5) Method according to clause 4 wherein the end-point reaction is carried out with an additional stage, the final step, of temperature profile of 80° C., 5 min.
6) A method for detecting infection by Severe acute respiratory syndrome coronavirus 2, interchangeable in that it comprises a detection method as defined in clause 3.
7) A reaction kit for detecting Severe acute respiratory syndrome coronavirus 2 infection, characterized in that it comprises a set of primers as defined in clause 1. and in clause 2.
8) Infection detection kit according to clause 7. characterized in that it contains 10.0 μl WarmStart LAMP KIT (RNA&DNA) (New England Biolabs).
9) Infection detection kit according to clauses 7 and/or 8, comprising amplification primers as defined in clause 1 and in clause 2, wherein the primers have the following concentrations: 0.13 μM F3, 0.13 μM B3, 1.00 μM FIP, 1.00 μM BIP, 0.25 μM LoopB; double-stranded DNA fluorescent marker-EvaGreen≤1×; or Fluorescent Dye≤0.4 μl; or Green Fluorescent Dye≤0,8 μl; or Syto-13≤16 μM or SYTO-82≤16 M; or other double-stranded DNA fluorescent dye at a concentration not inhibiting the amplification reaction.

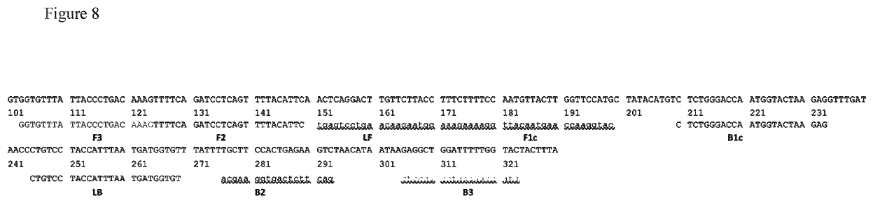

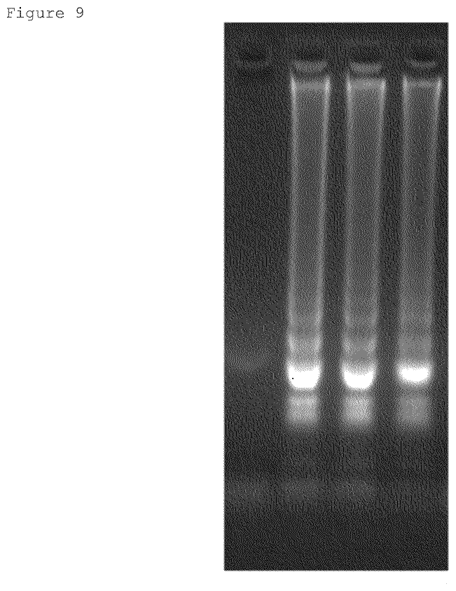

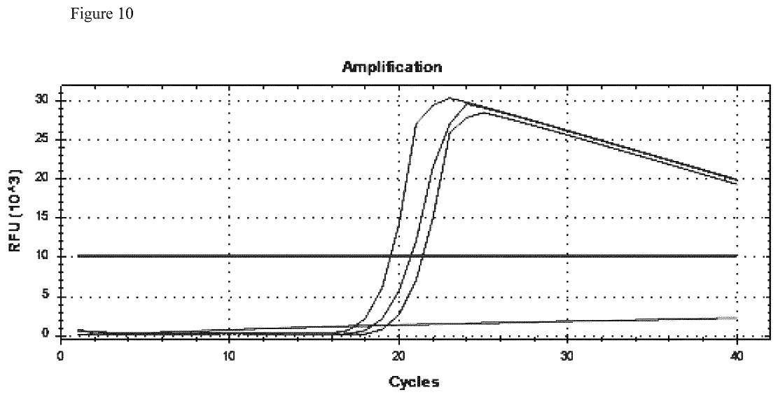

Example 6-S gene-Primer sequences The specific sequences of oligonucleotides used to detect Severe acute respiratory syndrome coronavirus 2 genetic material utilizing LAMP technology are shown in FIG. 8 and characterized below:
1. Oligonucleotide sequence SARS-COV-2 SF3: 5′ GGTGTTTATTACCCTGACAAAG3′ (SEQ ID NO: 15) is a sequence identical to the Severe acute respiratory syndrome coronavirus 2 S gene (strand 5′-3′), which from the 3′ end is adjacent to the F2 primer.
2. Oligonucleotide sequence SARS-COV-2 SB3: 5′ GTACCAAAAATCCAGCCTC3′ (SEQ ID NO: 16) is a complementary fragment of Severe acute respiratory syndrome coronavirus 2 S gene (strand 5′-3′) separated by 180 nucleotides from the 3′ end of oligonucleotide 1.
3. Oligonucleotide sequence SARS-COV-2 SF2: 5′ TTTTCAGATCCTCAGTTTTACATTC 3′ (SEQ ID NO: 17) is a sequence identical to the Severe acute respiratory syndrome coronavirus 2 S gene (strand 5′-3′) sequence immediately adjacent to the 3 ‘end of oligonucleotide 1.
4. Oligonucleotide sequence SARS-COV-2 SB2: 5’ GACTTCTCAGTGGAAGCA 3′ (SEQ ID NO: 18) is a complementary fragment of Severe acute respiratory syndrome coronavirus 2 S gene (strand 5′-3′) separated by 151 nucleotides from the 3′ end of oligonucleotide 1.
5. Oligonucleotide sequence SARS-COV-2 SF1c: 5′ CATGGAACCAAGTAACATTGGAAAA 3′ (SEQ ID NO: 19) is a complementary fragment of Severe acute respiratory syndrome coronavirus 2 S gene (strand 5′-3′) separated by 50 nucleotides from the 3′ end of oligonucleotide 1.
6. Oligonucleotide sequence SARS-COV-2 SB1c: 5′ CTCTGGGACCAATGGTACTAAGAG 3′ (SEQ ID NO: 20) is a sequence identical to the Severe acute respiratory syndrome coronavirus 2 S gene separated by 85 nucleotides from the 3′ end of oligonucleotide 1.
7. Oligonucleotide sequence SARS-COV-2 SLoopF: 5′ GAAAGGTAAGAACAAGTCCTGAGT 3′ (SEQ ID NO: 21).
8. Oligonucleotide sequence SARS-COV-2 SLoopB: 5′ CTGTCCTACCATTTAATGATGGTGT 3′ (SEQ ID NO: 22).

The F1c and F2 oligonucleotide sequences are preferably linked by a TTTT bridge and used in the form of a Forward Inner Primer (FIP); for example: 5′ CATGGAACCAAGTAACATTGGAAAANNNNTTTTCAGATCCTCAGTTTTACATTC 3′ (SEQ ID NO: 23); or most particularly 5′ CATGGAACCAAGTAACATTGGAAAATTTTTTTTCAGATCCTCAGTTTTACATTC 3′ (SEQ ID NO: 24)—where N is any nucleotide.

The B1c and B2 oligonucleotide sequences are preferably linked via a TTTT bridge and are used in the form of a Backward Inner Primer (BIP); for example: 5′ CTCTGGGACCAATGGTACTAAGAGNNNNGACTTCTCAGTGGAAGCA 3′ (SEQ ID NO: 25); or most particularly 5′ CTCTGGGACCAATGGTACTAAGAGTTTTGACTTCTCAGTGGAAGCA 3′ (SEQ ID NO: 26)—where N is any nucleotide.

### Example 7

Severe acute respiratory syndrome coronavirus 2 S gene amplification method utilizing LAMP technology with oligonucleotides characterized in Example 6, with the following composition of the reaction mixture:
10.0 μl WarmStart LAMP KIT (RNA&DNA)
0.13 μM F3
0.13 μM B3
1.06 μM FIP
1.06 μM BIP
0.27 μM LoopF
0.27 μM LoopB
   Fluorescent Dye Interacting with Double-Stranded DNA:
EvaGreen≤1× or Fluorescent dye 50× (New England Biolabs) at 0,4 μl, or Green Fluorescent Dye (Lucigen) at ≤0,8 μl, or
Syto-13≤16 μM or SYTO-82≤16 μM, or other fluorescent dye interacting with double-stranded DNA at a concentration that does not inhibit the amplification reaction.
   DNA template≥25 copies/reaction

### Example 8

Severe acute respiratory syndrome coronavirus 2 S gene amplification method using oligonucleotides characterized in Example 6 in LAMP technology with the composition of the reaction mixture characterized in Example 7 with the following temperature profile:
1) 60° C., 40 min
2) preferably at 80° C. end-point reactions, 5 min. Such end point reactions aim to denature the polymerase in order to stop amplification.

### Example 9

The method of amplification and detection of the Severe acute respiratory syndrome coronavirus 2 S gene using oligonucleotides characterized in Example 6 in LAMP technology with the composition of the reaction mixture characterized in Example 5 with the temperature profile characterized in Example 8 and the detection method described below.

Fluorescent dye has the ability to interact with double-stranded DNA present in the reaction mixture as described in Example 4 above.

### Example 10 Method Sensitivity

Sensitivity was determined by setting-up RealTime-RT-LAMP for a series of dilutions of the Twist Synthetic SARS-COV-2 RNA Control 2 (Twist Bioscience); with a minimum amount of 25 copies of viral RNA in the reaction mixture, where product was measured in a real time-FIG. 2 (RealTime-RT-LAMP for a series of dilutions).

The time after which it is possible to detect the emitted fluorescence corresponding to a specific SARS-COV-2 genome fragment for given copy number is shown in Table 2.

Conclusion: the characterized primers enable the detection of Severe acute respiratory syndrome coronavirus 2 by detecting a fragment of the S gene with a minimum of 25 copies/reaction mixture.

TABLE 2
Time needed to detect fluorescence for individual dilutions of
the Severe acute respiratory syndrome coronavirus 2 RNA standard
(Twist Synthetic SARS-CoV-2 RNA Control 2, Twist Bioscience).

| | Time of exceeding the fluorescence |
|---|---|
| Sample | baseline [min] |
| SARS-CoV-2 NTC | Undetermined |
| SARS-CoV-2 25 copies | 19.48 |
| SARS-CoV-2 50 copies | 20.68 |
| SARS-CoV-2 100 copies | 21.38 |

The superiority of the amplification method and set of primers (oligonucleotides) characterized in this patent over other tests based on the Real Time-RT-LAMP technology lies in its high sensitivity shown in FIG. 8 and the dramatic reduction of the analysis time shown in Table 2 when compared to standard RT-PCR techniques.

Accordingly, further embodiments are exemplified in the following clauses:
1. A primer set for detecting SARS-COV-2 using a reverse transcription loop-mediated isothermal amplification (RT-LAMP) method, comprising:
   i) at least a first F3 primer comprising a nucleotide sequence at least 90% identical to SEQ ID NO: 15;
   ii) at least a first B3 primer comprising a nucleotide sequence at least 90% identical to SEQ ID NO: 16;
   iii) at least a first F2 primer comprising a nucleotide sequence at least 90% identical to SEQ ID NO: 17;
   iv) at least a first B2 primer comprising a nucleotide sequence at least 90% identical to SEQ ID NO: 18;
   v) at least a first F1c primer comprising a nucleotide sequence at least 90% identical to SEQ ID NO: 19; and
   vi) at least a first B1c primer comprising a nucleotide sequence at least 90% identical to SEQ ID NO: 20.
2. The primer set according to clause 1, wherein:
   i) the first F3 primer comprises a nucleotide sequence at least 95% identical to SEQ ID NO: 15;
   ii) the first B3 primer comprises a nucleotide sequence at least 95% identical to SEQ ID NO: 16;
   iii) the first F2 primer comprises a nucleotide sequence at least 95% identical to SEQ ID NO: 17;
   iv) the first B2 primer comprises a nucleotide sequence at least 95% identical to SEQ ID NO: 18;
   v) the first F1c primer comprises a nucleotide sequence at least 95% identical to SEQ ID NO: 19; and/or
   vi) the first B1c primer comprises a nucleotide sequence at least 95% identical to SEQ ID NO: 20.
3. The primer set according to clause 1 or clause 2, wherein:
   i) the first F3 primer comprises a nucleotide sequence at least 98% identical to SEQ ID NO: 15;
   ii) the first B3 primer comprises a nucleotide sequence at least 98% identical to SEQ ID NO: 16;
   iii) the first F2 primer comprises a nucleotide sequence at least 98% identical to SEQ ID NO: 17;
   iv) the first B2 primer comprises a nucleotide sequence at least 98% identical to SEQ ID NO: 18;
   v) the first F1c primer comprises a nucleotide sequence at least 98% identical to SEQ ID NO: 19; and/or
   vi) the first B1c primer comprises a nucleotide sequence at least 98% identical to SEQ ID NO: 20.
4. The primer set according to any one of clauses 1 to 3, wherein:
   i) the first F3 primer comprises SEQ ID NO: 15 or a sequence resulting from a single nucleotide exchange, substitution or deletion of a single nucleotide;
   ii) the first B3 primer comprises SEQ ID NO: 16 or a sequence resulting from a single nucleotide exchange, substitution or deletion of a single nucleotide;
   iii) the first F2 primer comprises SEQ ID NO: 17 or a sequence resulting from a single nucleotide exchange, substitution or deletion of a single nucleotide;
   iv) the first B2 primer comprises SEQ ID NO: 18 or a sequence resulting from a single nucleotide exchange, substitution or deletion of a single nucleotide;
   v) the first F1c primer comprises SEQ ID NO: 19 or a sequence resulting from a single nucleotide exchange, substitution or deletion of a single nucleotide; and/or vi) the first B1c primer comprises SEQ ID NO: 20 or a sequence resulting from a single nucleotide exchange, substitution or deletion of a single nucleotide.
5. The primer set according to any one of clauses 1 to 4, wherein:
   i) the first F3 primer comprises a nucleotide sequence identical to SEQ ID NO: 15;
   ii) the first B3 primer comprises a nucleotide sequence identical to SEQ ID NO: 16;
   iii) the first F2 primer comprises a nucleotide sequence identical to SEQ ID NO: 17;
   iv) the first B2 primer comprises a nucleotide sequence identical to SEQ ID NO: 18;
   v) the first F1c primer comprises a nucleotide sequence identical to SEQ ID NO: 19; and/or
   vi) the first B1c primer comprises a nucleotide sequence identical to SEQ ID NO: 20.
6. The primer set according to any one of clauses 1 to 5, wherein:
   i) the first F3 primer consists of SEQ ID NO: 15;
   ii) the first B3 primer consists of SEQ ID NO: 16;
   iii) the first F2 primer consists of SEQ ID NO: 17;
   iv) the first B2 primer consists of SEQ ID NO: 18;
   v) the first F1c primer consists of SEQ ID NO: 19; and/or
   vi) the first B1c primer consists of SEQ ID NO: 20.
7. The primer set according to any one of clauses 1 to 6, further comprising:
   vii) a LoopF primer comprising a nucleotide sequence at least 90% identical to SEQ ID NO: 21; and
   viii) a LoopB primer comprising a nucleotide sequence at least 90% identical to SEQ ID NO: 22.
8. The primer set according to clause 7, wherein the LoopF primer:
   a) comprises a nucleotide sequence at least 95% identical to SEQ ID NO: 21;
   b) comprises a nucleotide sequence at least 98% identical to SEQ ID NO: 21;
   c) comprises a nucleotide sequence identical to SEQ ID NO: 21; or
   d) consists of SEQ ID NO: 21.
9. The primer set according to clause 7 or clause 8, wherein the LoopB primer:
   a) comprises a nucleotide sequence at least 95% identical to SEQ ID NO: 22;
   b) comprises a nucleotide sequence at least 98% identical to SEQ ID NO: 22;
   c) comprises a nucleotide sequence identical to SEQ ID NO: 22; or
   d) consists of SEQ ID NO: 22.
10. The primer set according to any one of clauses 1 to 9, wherein the F1c primer and the F2 primer are linked to form a Forward Inner Primer (FIP).
11. The primer set according to clause 10, wherein the F1c primer and the F2 primer are linked by a nucleotide bridge, preferably wherein the bridge is made of between one and ten nucleotides, more preferably wherein the bridge is TTTT.
12. The primer set according to clause 10, wherein the FIP primer has a sequence selected from SEQ ID Nos: 23 and 24.
13. The primer set according to any one of clauses 1 to 12, wherein the B1c primer and the B2 primer are linked to form a Backward Inner Primer (BIP).
14. The primer set according to clause 13, wherein the B1c primer and the B2 primer are linked by a nucleotide bridge, preferably wherein the bridge is made of between one and ten nucleotides, more preferably wherein the bridge is TTTT.
15. The primer set according to clause 13, wherein the BIP primer has a sequence selected from SEQ ID Nos: 25 and 26.
16. A kit for detecting Severe acute respiratory syndrome coronavirus 2 (SARS-COV-2) in a sample, the kit comprising:
   reagents for amplifying RNA in a sample using a RT-LAMP technique; and
   a primer set according to any one of clauses 1 to 15.
17. The kit according to clause 16, wherein one or more of the reagents or primers is dried or lyophilised; preferably wherein all of the primers are dried or lyophilised.
18. A method of detecting SARS-COV-2 in a sample, comprising: amplifying by isothermal amplification at least a portion of the SARS-COV-2 genome using a primer set according to any one of clauses 1 to 15.
19. The method according to clause 18, further comprising detecting the amplified product by observing a fluorescence signal coming from the sample.
20. The method according to clause 18 or clause 19, wherein the sample is obtained from a patient.
21. The method according to any one of clauses 18 to 20, wherein the amplification is carried out at about 60° C.
22. The method according to any one of clauses 18 to 21, wherein the amplification is carried out for 8-45 minutes, preferably for about 40 minutes.
23. The method according to any one of clauses 18 to 22, further comprising a final step of holding at a temperature of between 75 and 85° C. for 3 to 10 minutes, preferably at a temperature of about 80° C. for about 5 minutes.
24. The method according to any one of clauses 18 to 23, wherein the primers are used at the following reaction mixture concentrations:
   0.10 to 0.15 μM F3, preferably about 0.13 μM;
   0.10 to 0.15 μM B3, preferably about 0.13 μM;
   0.80 to 1.2 μM FIP, preferably about 1.06 M;
   0.80 to 1.2 μM BIP, preferably about 1.06 μM;
   0.2 to 0.3 μM LoopF, preferably about 0.27 μM; and/or
   0.2 to 0.3 μM LoopB, preferably about 0.27 μM.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

1. A primer set for detecting SARS-COV-2 using a reverse transcription loop-mediated isothermal amplification (RT-LAMP) method, comprising:
i) at least a first F3 primer comprising a nucleotide sequence at least 90% identical to SEQ ID NO: 1;
ii) at least a first B3 primer comprising a nucleotide sequence at least 90% identical to SEQ ID NO: 2;
iii) at least a first F2 primer comprising a nucleotide sequence at least 90% identical to SEQ ID NO: 3;
iv) at least a first B2 primer comprising a nucleotide sequence at least 90% identical to SEQ ID NO: 4;
v) at least a first F1c primer comprising a nucleotide sequence at least 90% identical to SEQ ID NO: 5 or to SEQ ID NO: 8; and
vi) at least a first B1c primer comprising a nucleotide sequence at least 90% identical to SEQ ID NO: 6.

2. The primer set according to claim 1, wherein:
i) the first F3 primer comprises a nucleotide sequence at least 95% identical to SEQ ID NO: 1;
ii) the first B3 primer comprises a nucleotide sequence at least 95% identical to SEQ ID NO: 2;
iii) the first F2 primer comprises a nucleotide sequence at least 95% identical to SEQ ID NO: 3;
iv) the first B2 primer comprises a nucleotide sequence at least 95% identical to SEQ ID NO: 4;
v) the first F1c primer comprises a nucleotide sequence at least 95% identical to SEQ ID NO: 5 or to SEQ ID NO: 8; and/or
vi) the first B1c primer comprises a nucleotide sequence at least 95% identical to SEQ ID NO: 6.

3. The primer set according to claim 2, wherein:
i) the first F3 primer comprises a nucleotide sequence at least 98% identical to SEQ ID NO: 1;
ii) the first B3 primer comprises a nucleotide sequence at least 98% identical to SEQ ID NO: 2;
iii) the first F2 primer comprises a nucleotide sequence at least 98% identical to SEQ ID NO: 3;
iv) the first B2 primer comprises a nucleotide sequence at least 98% identical to SEQ ID NO: 4;
v) the first F1c primer comprises a nucleotide sequence at least 98% identical to SEQ ID NO: 5 or to SEQ ID NO: 8; and/or
vi) the first B1c primer comprises a nucleotide sequence at least 98% identical to SEQ ID NO: 6.

4. The primer set according to claim 3, wherein:
i) the first F3 primer comprises SEQ ID NO: 1 or a sequence resulting from a single nucleotide exchange, substitution or deletion of a single nucleotide;
ii) the first B3 primer comprises SEQ ID NO: 2 or a sequence resulting from a single nucleotide exchange, substitution or deletion of a single nucleotide;
iii) the first F2 primer comprises SEQ ID NO: 3 or a sequence resulting from a single nucleotide exchange, substitution or deletion of a single nucleotide;
iv) the first B2 primer comprises SEQ ID NO: 4 or a sequence resulting from a single nucleotide exchange, substitution or deletion of a single nucleotide;
v) the first F1c primer comprises SEQ ID NO: 5 or SEQ ID NO: 8 or a sequence resulting from a single nucleotide exchange, substitution or deletion of a single nucleotide; and/or
vi) the first B1c primer comprises SEQ ID NO: 6 or a sequence resulting from a single nucleotide exchange, substitution or deletion of a single nucleotide.

5. The primer set according to claim 4, wherein:
i) the first F3 primer comprises a nucleotide sequence identical to SEQ ID NO: 1;
ii) the first B3 primer comprises a nucleotide sequence identical to SEQ ID NO: 2;
iii) the first F2 primer comprises a nucleotide sequence identical to SEQ ID NO: 3;
iv) the first B2 primer comprises a nucleotide sequence identical to SEQ ID NO: 4;
v) the first F1c primer comprises a nucleotide sequence identical to SEQ ID NO: 5 or to SEQ ID NO: 8; and/or
vi) the first B1c primer comprises a nucleotide sequence identical to SEQ ID NO: 6.

6. The primer set according to claim 5, wherein:
i) the first F3 primer consists of SEQ ID NO: 1;
ii) the first B3 primer consists of SEQ ID NO: 2;
iii) the first F2 primer consists of SEQ ID NO: 3;
iv) the first B2 primer consists of SEQ ID NO: 4;
v) the first F1c primer consists of SEQ ID NO: 5 or SEQ ID NO: 8; and/or
vi) the first B1c primer consists of SEQ ID NO: 6.

7. The primer set according to claim 1, further comprising:
vii) a LoopB primer comprising a nucleotide sequence at least 90% identical to SEQ ID NO: 7.

8. The primer set according to claim 7, wherein the first LoopB primer:
a) comprises a nucleotide sequence at least 95% identical to SEQ ID NO: 7;
b) comprises a nucleotide sequence at least 98% identical to SEQ ID NO: 7;
c) comprises a nucleotide sequence identical to SEQ ID NO: 7; or
d) consists of SEQ ID NO: 7.

9. The primer set according to claim 1, wherein the F1c primer and the F2 primer are linked to form a Forward Inner Primer (FIP).

10. The primer set according to claim 9, wherein the F1c primer and the F2 primer are linked by a nucleotide bridge.

11. The primer set according to claim 9, wherein the FIP primer has a sequence selected from SEQ ID Nos: 9, 10 or 11.

12. The primer set according to claim 1, wherein the B1c primer and the B2 primer are linked to form a Backward Inner Primer (BIP).

13. The primer set according to claim 12, wherein the B1c primer and the B2 primer are linked by a nucleotide bridge.

14. The primer set according to claim 12, wherein the BIP primer has a sequence selected from SEQ ID Nos: 12 and 13.

15. A kit for detecting Severe acute respiratory syndrome coronavirus 2 (SARS-COV-2) in a sample, the kit comprising:
reagents for amplifying RNA in a sample using a RT-LAMP technique; and
a primer set according to claim 1.

16. The kit according to claim 15, wherein one or more of the reagents or primers is dried or lyophilised.

17. A method of detecting SARS-COV-2 in a sample, comprising: amplifying by isothermal amplification at least a portion of the SARS-COV-2 genome using a primer set according to claim 1.

18. The method according to claim 17, further comprising detecting the amplified product by observing a fluorescence signal coming from the sample.

19. The method according to claim 17, wherein the sample is obtained from a patient.

20. The method according to claim 17, wherein the amplification is carried out at between 65 and 70° C..

21. The method according to claim 17, wherein the amplification is carried out for 8-45 minutes.

22. The method according to claim 17, further comprising a final step of holding at a temperature of between 75 and 85° C. for 3 to 10 minutes.

23. The method according to claim 17, wherein the primers are used at reaction mixture concentrations selected from the group consisting of:
0.10 to 0.15 μM F3, preferably about 0.13 μM;
0.10 to 0.15 μM B3, preferably about 0.13 μM;
0.80 to 1.2 μM FIP, preferably about 1.0 μM;
0.80 to 1.2 μM BIP, preferably about 1.00 M; and/or
0.2 to 0.3 μM LoopB, preferably about 0.25 μM.


SEQUENCE LISTING (29 sequences)

SEQ ID NO: 1 - DNA, 18 bp, Artificial Sequence
  primer SARS-CoV-2 NF3
cggcagtcaagcctcttc

SEQ ID NO: 2 - DNA, 20 bp, Artificial Sequence
  primer SARS-CoV-2 NB3
ttgctctcaagctggttcaa

SEQ ID NO: 3 - DNA, 20 bp, Artificial Sequence
  primer SARS-CoV-2 NF2
cgttcctcatcacgtagtcg

SEQ ID NO: 4 - DNA, 20 bp, Artificial Sequence
  primer SARS-CoV-2 NB2
tctgtcaagcagcagcaaag

SEQ ID NO: 5 - DNA, 20 bp, Artificial Sequence
  primer SARS-CoV-2 NF1c
tcsyytactgctgcctggag

SEQ ID NO: 6 - DNA, 22 bp, Artificial Sequence
  primer SARS-CoV-2 NB1c
tctcctgctagaatggctggca

SEQ ID NO: 7 - DNA, 19 bp, Artificial Sequence
  primer SARS-CoV-2 NLoopB
atggcggtgatgctgctct

SEQ ID NO: 8 - DNA, 20 bp, Artificial Sequence
  primer SARS-CoV-2 NF1c′
tcccctactgctgcctggag

SEQ ID NO: 9 - DNA, 44 bp, Artificial Sequence
  primer SARS-CoV-2 FIP-1
  misc_feature 21..24: where ′n′ is any nucleotide
tcsyytactgctgcctggagnnnncgttcctcatcacgtagtcg

SEQ ID NO: 10 - DNA, 44 bp, Artificial Sequence
  primer SARS-CoV-2 FIP-2
tcsyytactgctgcctggagttttcgttcctcatcacgtagtcg

SEQ ID NO: 11 - DNA, 44 bp, Artificial Sequence
  primer SARS-CoV-2 FIP-3
tcccctactgctgcctggagttttcgttcctcatcacgtagtcg

SEQ ID NO: 12 - DNA, 46 bp, Artificial Sequence
  primer SARS-CoV-2 BIP-1
  misc_feature 23..26: where ′n′ is any nucleotide
tctcctgctagaatggctggcannnntctgtcaagcagcagcaaag

SEQ ID NO: 13 - DNA, 46 bp, Artificial Sequence
  primer SARS-CoV-2 BIP-2
tctcctgctagaatggctggcatttttctgtcaagcagcagcaaag

SEQ ID NO: 14 - DNA, 1260 bp, Betacoronavirus severe acute respiratory syndrome- related coronavirus 2
  N gene of Severe acute respiratory syndrome coronavirus 2
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 15 - DNA, 22 bp, Artificial Sequence
  primer SARS-CoV-2 SF3SARS-CoV-2 SF3
ggtgtttattaccctgacaaag

SEQ ID NO: 16 - DNA, 19 bp, Artificial Sequence
  primer SARS-CoV-2 SB3
gtaccaaaaatccagcctc

SEQ ID NO: 17 - DNA, 25 bp, Artificial Sequence
  primer SARS-CoV-2 SF2
ttttcagatcctcagttttacattc

SEQ ID NO: 18 - DNA, 18 bp, Artificial Sequence
  primer SARS-CoV-2 SB2
gacttctcagtggaagca

SEQ ID NO: 19 - DNA, 25 bp, Artificial Sequence
  primer SARS-CoV-2 SF1c
catggaaccaagtaacattggaaaa

SEQ ID NO: 20 - DNA, 24 bp, Artificial Sequence
  primer SARS-CoV-2 SB1c
ctctgggaccaatggtactaagag

SEQ ID NO: 21 - DNA, 24 bp, Artificial Sequence
  primer SARS-CoV-2 SLoopF
gaaaggtaagaacaagtcctgagt

SEQ ID NO: 22 - DNA, 25 bp, Artificial Sequence
  primer SARS-CoV-2 SLoopB
ctgtcctaccatttaatgatggtgt

SEQ ID NO: 23 - DNA, 54 bp, Artificial Sequence
  primer SARS-CoV-2 FIP-1
  misc_feature 26..29: where ′n′ is any nucleotide
catggaaccaagtaacattggaaaannnnttttcagatcctcagttttacattc

SEQ ID NO: 24 - DNA, 54 bp, Artificial Sequence
  primer SARS-CoV-2 FIP-2
catggaaccaagtaacattggaaaattttttttcagatcctcagttttacattc

SEQ ID NO: 25 - DNA, 46 bp, Artificial Sequence
  primer SARS-CoV-2 BIP-1
  misc_feature 25..28: where ′n′ is any nucleotide
ctctgggaccaatggtactaagagnnnngacttctcagtggaagca

SEQ ID NO: 26 - DNA, 46 bp, Artificial Sequence
  primer SARS-CoV-2 BIP-2
ctctgggaccaatggtactaagagttttgacttctcagtggaagca

SEQ ID NO: 27 - DNA, 3822 bp, Betacoronavirus severe acute respiratory syndrome- related coronavirus 2
  S gene of Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 28 - DNA, 170 bp, Coronaviridae
  Portion of N gene of Severe acute respiratory syndrome coronavirus 2
aggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaa

SEQ ID NO: 29 - DNA, 230 bp, Coronaviridae
  Portion of S gene of Severe acute respiratory syndrome coronavirus 2
gtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactacttta